(19) United States
(12) Patent Application Publication
(10) Pub. No.: US 2009/0092565 A1
(43) Pub. Date: Apr. 9, 2009

(54) DENTAL ENAMEL RECALCIFICATION ACCELERATOR AND CONTAINING THE SAME, ORAL COMPOSITION AND FOOD OR BEVERAGE

(75) Inventors: Takaaki Koyama, Saitama (JP); Daigo Sugita, Saitama (JP); Shinya Asada, Saitama (JP); Susumu Shimura, Saitama (JP)

Correspondence Address: Young & Thompson, 745 S. 23rd Street., Second Floor, Arlington, VA, 22202, US

(73) Assignee: LOTTE CO., LTD., Tokyo (JP)

(21) Appl. No.: 11/914,877

(22) PCT Filed: May 24, 2005
(86) PCT No.: PCT/JP2005/009466

§ 371 Date: Feb. 13, 2008

(30) Foreign Application Priority Data
May 19, 2005 (JP) 2005-147109

(51) Int. Cl. A61K 8/73; A61Q 11/00

(52) U.S. Cl. 424/57; 424/49

(57) ABSTRACT

A dental enamel recalcification accelerator that even when used in oral compositions and food or beverage, poses no safety problem, and that is capable of effectively promoting the recalcification of decalcified dental enamel to thereby positively suppress any dental caries; and containing the same, an oral composition and food or beverage. There is provided a dental enamel recalcification accelerator comprising anhydrogalactose-containing red algae and/or an extract thereof as an active ingredient. Further, there are provided an oral composition and food or beverage comprising the dental enamel recalcification accelerator.

### TECHNICAL FIELD OF THE INVENTION

The present invention relates to dental enamel recalcification accelerator, and an oral composition and a food or beverage containing the same.

### BACKGROUND ART

In general, a dental caries begins with the following processes: oral streptococcus (caries bacteria) such as Streptococcus mutans and Streptococcus sobrinus adhere to the tooth surface; glucan is generated by the agency of glucosyltransferase enzyme contained in these bacteria; and the glucan forms plaque. As said bacterial metabolize sugar and starch in food residues, an acid is generated in the plaque, the acid decalcifying the dental enamel, creating what is called the initial stage of caries.

Saliva includes calcium and phosphate, both having a function of repairing and recalcifying said decalcified portion of the teeth to restore them to their original condition. Two opposite phenomena of decalcification and recalcification are always occurring on the tooth surface, the necessary balance therebetween usually being maintained. However, said balance is inclined to decalcification as plaque increases, resulting in development of dental caries.

The crystal that forms enamel on the tooth surface is a hexagonal hydroxyapatite Ca₁₀(PO₄)₆OH₂ that comprises calcium phosphate. It may be said that at the initial stage of caries, the decalcification is a dissolution of inorganic components of dental enamel while the recalcification is a restoration and regrowth of the calcium phosphate crystal that has survived the dissolution.

In order to prevent dental caries, some agents have beer developed such as tooth adhesion inhibitor and antibacterial agent; against caries bacteria, as well as glucosyltransferase enzyme inhibitor that suppresses glucan production caused by the caries bacteria. However, an antibacterial agent is not a specific maternal having antibacterial effect solely on caries bacteria, which poses safety problems, and a glucosyltransferase enzyme inhibitor is liable to be affected by saliva.

A caries prevention composition is known, into which hydroxyapatite and fluoride, both, having a crystalline structure similar to that of inorganic components of teeth, are blended, the caries prevention composition recalcifying the decalcified surfaces of a tooth (sec, for example, Patent Document 1). This, however, poses safety problems to blend fluorides such as sodium fluoride, sodium monofluorophosphate and stannous fluoride into an oral composition or a food or beverage.

Also known is a recalcification method in which microparticles of hydroxyapatite are used in combination with xylitol to thereby recalcify the decalcified dental enamel (see, for example, Patent Document 2). An industrially-produced hydroxyapatite has a poor reactivity due to its own properties as a stable composition. To be more specific, said industrially-produced hydroxyapatite is different in terms of crystalline structure from the hydroxyapatite that comprises a tooth of a living organism, resulting in insufficient recalcification effect.

Also known is an oral composition that contains liquefied calcium phosphate (see, for example, Patent Document 3). The calcium phosphate alone does not provide sufficient recalcification effect.

And also known is a dental enamel recalcification accelerator that contains seaweeds, in particular, a glue plant belonging to the red algae Cryptonemiales (see, for example, Patent Documents 4 and 5). The stability of said dental enamel recalcification promoting effect is dependent on the type of the seaweed, resulting in insufficient recalcification effect. It has not been realized that red algae, especially the anhydrogalactose-containing red algae, and/or extract thereof, such as at least one polysaccharide selected from the group consisting of κ-carrageenan, ι-carrageenan and Furcelleran, have, in particular, the dental enamel recalcification promoting effect.

Patent Document 1: Patent Publication No. H2-31049
Patent Document 2: Laid-open Patent Publication No. H9-175963
Patent Document 3: Laid-open Patent Publication No. H8-319224

### Patent Document 4: Laid-open Patent Publication No. 2000-53549

### Patent Document 5: Laid-open Patent Publication No. 2000-128752

### SUMMARY OF THE INVENTION

### Problem to be Solved

Considering the above background, the present invention is aimed to provide a recalcification accelerator that even when used in oral compositions and food or beverage, poses no safety problem, and that is capable of effectively accelerating the recalcification of decalcified dental enamel to thereby positively suppress any dental caries; and an oral composition and food or beverage containing the same.

### Means of Solving Problems

The inventors of the present invention have been diligently committed to research, and found that the object could be achieved by means of red algae, in particular, anhydrogalactose-containing red algae, and/or an extract thereof; and the present invention is completed.

The dental enamel recalcification accelerator according to the present invention comprises anhydrogalactose-containing red algae and/or an extract thereof as an active ingredient; or alternatively, comprises the anhydrogalactose-containing red algae and/or an extract thereof, together with xylitol and dicalcium phosphate as active ingredients. The anhydrogalactose include at least one polysaccharide selected from the group consisting of κ-carrageenan, ι-carrageenan and Furcelleran, especially κ-carrageenan, which provides a remarkable and preferable effect.

In other words, the present invention relates to said dental enamel recalcification accelerator comprising, as an active ingredient, anhydrogalactose-containing red algae and/or an extract thereof, and, according to the necessity, xylitol and/or dicalcium phosphate; and an oral composition and a food or beverage containing the same, including the oral composition and the food or beverage having an indication attached to the effect that recalcification of teeth will be enhanced.

### ADVANTAGEOUS EFFECT OF THE INVENTION

The dental enamel recalcification accelerator according to the present, invention comprises anhydrogalactose-containing red algae and/or an extract thereof as an active ingredient; or alternatively, comprises anhydrogalactose-containing red algae and/or an extract thereof in combination with xylitol and dicalcium phosphate as an active ingredient; the accelerator posing no safety problem even when used in oral compositions and food or beverage, and being capable of effectively promoting the recalcification of decalcified dental enamel to thereby positively suppress any dental caries.

In particular, the effect is highly remarkable when at least one polysaccharide selected from the group that consisting of κ-carrageenan, ι-carrageenan and Furcelleran, especially κ-carrageenan, as an extract of anhydrogalactose-containing red algae, is used in combination with xylitol and dicalcium phosphate. In other words, an intense recalcification that starts from the surface layer of teeth caused by an extract of anhydrogalactose-containing red algae (at least one polysaccharide selected from the group consisting of κ-carrageenan, ι-carrageenan and Furcelleran, especially κ-carrageenan), in combination with a recalcification that starts from the deep layer of teeth caused by xylitol, enhances the dental enamel recalcification promoting effect more significantly than red algae that do not contain any anhydrogalactose.

### BEST MODE FOR CARRYING OUT THE INVENTION

The anhydrogalactose-containing red algae in the present invention selectively includes Bangiales, Porphyridiales, Goniotrichales, Compsopogonales, Nemaliales, Gelidiales, Cryptonemiales, Gigartinales, Palmariales and Ceramiales. Especially remarkable and preferable effect can be observed in Chondrus ocellatus Holmes and Gigartinales of Gigartinaceae family, and Eucheuma denticulatum of Solieriaceae family, each containing at least one polysaccharide selected from the group consisting of κ-carrageenan, ι-carrageenan and Furcelleran.

For these anhydrogalactose-containing red algae in the present invention, either the red algae shredded or powdered after necessary drying treatment, and the red algae extracted to be shredded or powdered can be used either separately or in combination. Another sea weed can be used together, which, though, is not expected to have as much effect as anhydrogalactose-containing red algae.

Furcelleran, κ-carrageenan and ι-carrageenan are effective as an extract of red algae according to the present invention. Non-limiting examples of the method of obtaining a red algae extract include the use of water or an organic solvent, in particular, an organic solvent that is compatible with water. Said extract can be available after being further fractionated and refined by means of an organic solvent and column chromatography.

Furthermore, said red algae and/or the extract thereof can also be available after being formed into emulsion, wettable powder, dust formulation or tablet by way of the following process: the red algae and/or the extract is dissolved or dispersed into an appropriate liquid carrier; or alternatively, the red algae and/or the extract is mixed with, or absorbed in, an appropriate powder carrier; and, as the case may be, agents such as emulsifier, dispersant, suspension agent, spreading agent, penetrant, moistening agent and stabilizer are added to the red algae and/or the extract.

Said red algae and/or the extract thereof enable satisfactory promotion of the dental enamel recalcification even when used alone in oral compositions and food or beverage. Much further promotion of the re calcification can be achieved much further in combination with xylitol and/or dicalcium phosphate.

In the dental enamel recalcification accelerator according to the present invention, the mixture ratio among red algae and/or an extract thereof, xylitol, and dicalcium phosphate is preferably 0.02-5.0:50:0.02-2.0, more preferably, 0.05-0.2:50:0.1-0.4.

Said recalcification accelerator, namely, an oral composition that contains red algae and/or an extract thereof, or in addition thereto, xylitol and/or dicalcium phosphate, includes dentifrices such as toothpaste, tooth powder and dental rinse; mouth washes; gingival massage cream; gargle tablet; and trochiscus. Said food or beverage includes confectionery such as chewing gum, candy, tablet, gummy jelly, chocolate, biscuit and snack; frozen dessert such as ice cream and sherbet; beverage; bread; hot cake; dairy product; meat pro duct such as ham and sausage; fish meat product such as steamed fish paste and fish sausage; prepared food; pudding; soup; and jam.

Blending of the dental enamel recalcification accelerator according to the present invention into the daily-used oral composition or the daily-consumed food or beverage enables daily consumption of the recalcification accelerator, allowing prevention of dental caries to be conducted in an easy and carefree manner. Also, blending of the recalcification accelerator into the oral composition or the food or beverage provides additional benefits such as longer staying time of the recalcification accelerator in the mouth, enabling the recalcification accelerator to spread all over the oral cavity, which is particularly preferable. More specifically, blending of the dental enamel recalcification accelerator into trochiscus, chewing gum, candy, tablet, gummy jelly, chocolate, ice cream, sherbet, frozen dessert, toothpaste, tooth powder, and gingival massage cream provides longer staying time of the recalcification accelerator in the mouth, which is remarkably preferable. Blending of the recalcification accelerator into other foods causes the recalcification accelerator to be contained in food residues stuck in between teeth, which is also remarkably preferable. Furthermore, blending of the recalcification accelerator into the beverage, soup, tooth liquid or mouth washes causes the recalcification accelerator to spread all over the oral cavity (including interdentium), which is remarkably preferable.

The amount of red algae and/or the extract thereof to be added to the oral composition or the food or beverage is preferably 0.01.-10.0% by weight. The amount of xylitol and dicalcium phosphate, each to be used in combination with red algae and/or the extract thereof, is preferably 1-95% by weight and 0.01-5.0% by weight, respectively, although it is hard to categorically determine the amount, which depends on the type and form of the oral composition or the food or beverage to be taken.

In the present invention, red algae and/or the extract thereof, xylitol and dicalcium phosphate can be added to the oral composition or the food or beverage at any stage of the manufacturing process of such products; and can also be mixed with residual raw material. In case the red algae and/or the extract thereof is used together with xylitol and dicalcium phosphate, all of these substances can be separately added, or can be premixed altogether to be added, to said oral composition or said food or beverage.

### WORKING EXAMPLES

The working examples and experimental examples according to the present invention are given below, but they do not limit the scope of the present invention.

### Experimental Example 1

### Effect of Dental Enamel Recalcification Accelerator in the Present Invention

The experiment to ascertain the effect of the dental enamel recalcification accelerator in the present invention was conducted as shown below with reference to the experiment method for ascertaining the effect of recalcification acceleration using extracted human teeth, which is described in the Shikwagakuho (the Journal of the Tokyo Dental College Society) Vol. 89, No. 9, 1.441-1455 (1989). κ-carrageenan, ι-carrageenan, Furcelleran and λ-carrageenan, made by Kyoktuto Kagaku Sangyou Co., Ltd., were used as an extract of anhydrogalactose-containing red algae polysaccharide.

The entire surface of the enamel block of the extracted human teeth was coated with sticky wax, with a window portion thereon of 3 by 4 mm in size left uncoated. The block was immersed in a 0.01 M acetic acid-sodium acetate buffer solution heated up to 50° C. (pH4.0) for two days, to form decalcified layers (see FIG. 1). Then, half of the window portion was coated with wax to prepare an experimental dental enamel block.

The recalcification treatment involved 1 mM CaCl₂, 0.6 mM KH₂PO₄ and 100 mM NaCl. A recalcification solution was used that had been prepared to be pH7.3 using 50 mM KOH solution to prepare ten types of solutions from A) to J) as described below, each held at 37° C., one piece of experimental dental enamel block being immersed in each solution for two weeks, respectively, and each solution being replaced with new one every three days.

A) a recalcification solution;
B) a recalcification solution containing 5% by weight of xylitol and 0.02% by weight of dicalcium phosphate;
C) a recalcification solution containing 0.05% by weight of κ-carrageenan;
D) a recalcification solution containing 0.05% by weight of κ-carrageenan, 5% by weight of xylitol and 0.02% by weight of dicalcium phosphate;
E) a recalcification solution containing 0.05% by weight of λ-carrageenan;
F) a recalcification solution containing 0.05% by weight of λ-carrageenan, 5% by weight of xylitol and 0.02% by weight of dicalcium phosphate;
G) a recalcification solution containing 0.05% by weight of ι-carrageenan;
H) a recalcification solution containing 0.05% by weight of ι-carrageenan, 5% by weight of xylitol and 0.02% by weight of dicalcium phosphate;
I) a recalcification solution containing 0.05% by weight of Furcelleran; and
J) a recalcification solution containing 0.05% by weight of Furcelleran, 5% by weight of xylitol and 0.02% by weight of dicalcium phosphate

After the recalcification treatment, the wax on each experimental dental enamel block was eliminated; the block was embedded with polyester resin (Regolac resin); an abrasive section with a diameter of 100 μm was created on the block; and a radiograph thereof was taken using a contact micro radiogram (CMR). Radiographing condition was 10 kV, 3 mA, exposure time being 30 minutes, the radiograph being taken concurrently with an aluminum step wedge as a criterion. The radiograph was developed according to the usual method.

Conditions of the recalcification that were revealed as a result of microradiography (MR) were visually evaluated to be classified into the following five levels:

Recalcification Level 0): No recalcification observed in the entire decalcified enamel layer;
Recalcification Level 1): Marginal recalcification observed in the decalcified enamel surface layer;
Recalcification Level 2): Relatively intense recalcification observed in the decalcified enamel surface layer; or recalcification observed in the decalcified enamel surface and deep layers;
Recalcification Level 3): Recalcification observed from the surface layer to the deep layer of the entire decalcified enamel layer;
Recalcification Level 4): Intense recalcification observed from the surface layer to the deep layer of the entire decalcified enamel layer;

FIGS. 1 to 5 below illustrate the results of the microradiography (MR) immediately after a decalcified layer was formed in the experimental dental enamel block, and the results of the microradiography (MR) after the Decalcification treatments.

FIG. 1 shows the MR immediately after the decalcification treatment; no recalcification observed (Recalcification level 0).

FIG. 2 shows the MR after the treatment with a recalcification solution A); marginal recalcification observed in the entire decalcified surface (Recalcification Level 1).

FIG. 3 shows the MR after the treatment with a recalcification solution B); recalcification observed in the surface layer, as well as the deep layer of the decalcified enamel layer (Recalcification Level 2).

FIG. 4 shows the MR after the treatment with a recalcification solution C); intense recalcification observed in the decalcified enamel surface layer (Recalcification Level 2).

FIG. 5 shows the MR after the treatment with a recalcification solution D); intense recalcification observed in the surface and deep layers of the entire decalcified enamel layer (Recalcification level 4).

FIG. 6 shows the MR after the treatment with a recalcification solution E); marginal recalcification observed in the entire decalcified enamel surface (Recalcification Level 1).

FIG. 7 shows the MR after the treatment with a recalcification solution F); recalcification observed in the surface layer, as well as the deep layer, of the decalcified enamel layer (Recalcification Level 2).

FIG. 8 shows the MR after the treatment with a recalcification solution G); intense recalcification observed in the decalcified enamel surface layer (Decalcification Level 2).

FIG. 9 shows the MR after the treatment with a recalcification solution H); intense recalcification observed in the surface and deep layers of the entire decalcified enamel layer (Recalcification Level 4).

FIG. 10 shows the MR after the treatment with a recalcification solution I); intense recalcification observed in the decalcified enamel surface layer (Recalcification Level 2).

FIG. 11 shows the MR after the treatment with a recalcification solution J); intense recalcification observed in the surface and deep layers of the entire decalcified enamel layer (Recalcification Level 4).

According to these results, it was observed that a polysaccharide, contained in a red algae extract, has a significantly more intense recalcification promoting effect when it contains anhydrogalactose than otherwise; and that combination thereof with xylitol and dicalcium phosphate remarkably enhances said recalcification promoting effect.

### Experimental Example 2

### Dental Enamel Recalcification Promoting Effect of Food or Beverage (Chewing Gum) in the Present Invention

Several types of chewing gum were prepared for each Example in accordance with the mixing ratio shown in Table 1, the gum comprising κ-carrageenan, ι-carrageen an and Furcelleran as an extract of the anhydrogalactose-containing red algae polysaccharide, and further in combination with xylitol and dicalcium phosphate:

Working Examples 1 to 3 (0.10% by weight of κ-carrageenan, ι-carrageenan and Furcelleran added, respectively);

Comparative Example 1 (no red algae extract added);

Comparative Example 2 (0.1% by weight of λ-carrageenan added).

TABLE 1

| | | Working |
|---|---|---|
| | | Examples 1 to 3, |
| | Comparative | Comparative |
| | Example 1 | Example 2 |
| Gum base | 28.0% by weight | 28.0% by weight |
| Xylitol | 41.0% by weight | 41.0% by weight |
| Palatinit | 25.0% by weight | 25.0% by weight |
| Maltitol | 4.7% by weight | 4.7% by weight |
| Softening agent | 0.8% by weight | 0.8% by weight |
| Dicalcium phosphate | 0.2% by weight | 0.2% by weight |
| Red algae polysaccharide | 0% by weight | 0.1% by weight |
| Purified water | 0.3% by weight | 0.2% by weight |

The recalcification promoting effect of the chewing gum of Working Examples 1 to 3 and Comparative Examples 1 and 2 was evaluated in the manner described below. The operation of extracting active ingredients from chewing gum was conducted with reference to the “Basic Research for Comprehensively Evaluating Cariogenicity of Foods and Sugar Substitutes (Problem No. 04304045): Report of Scientific Research Results Supported by Grant-in-Aid Program for Scientific Research (KAKENHI) for Academic Year 1993 (Research Leader: Tadashi Yamada); pp. 86-89”.

Each chewing gum for said Working Examples 1 to 3 and Comparative Example 1 and 2 was splintered to form a 10 g specimen, respectively, by means of weighing. Each specimen was mixed with 50 ml of recalcification solution (60° C.) of the composition equal to the one used in Experimental Example 1. The mixture was ground well with a glass rod to elute the contained ingredients, which were further mixed with another 50 ml of said recalcification solution (60° C.) for another elution operation. After the operation, small gum base was eliminated by centrifugal separation to obtain five types of chewing gum extract corresponding to each type of chewing gum to be used in Working Examples 1 to 3 and Comparative Examples 1 and 2, respectively.

Three pieces of experimental dental enamel block were immersed in each of the five types of chewing gum extract at 37° C. for two weeks, respectively, each extract being replaced with a new one every three days during the period. Then, the microradiographical (MR) results of each experimental dental enamel block were visually evaluated to be classified into five levels of recalcification, similarly to the Experimental Example 1 of the recalcification promoting effect. The degrees of recalcification in said Working Examples 1 to 3 and Comparative Examples 1 and 2 were calculated as an average value among the three experimental dental enamel blocks for each Example, respectively, the value being graphically indicated in FIG. 12.

The chewing gum in Comparative Example 1 (no red algae extract added) showed the recalcification degree of 1.67, while the chewing gum in Working Example 1 (0.1% of κ-carrageenan added) showed the recalcification degree of 2.67, the chewing gum in Working Example 2 (0.1% of ι-carrageenan added) showed the recalcification degree of 2.33, and the chewing gum in Working Example 3 (0.1% of Furcelleran added) showed the recalcification degree of 2.00. The chewing gum in Comparative Example 2 (0.1% of λ-carrageenan added) showed the recalcification degree of 1.67. This result proves that a red algae polysaccharide has a more intense recalcification promoting effect when it contains anhydrogalactose than otherwise.

### Experimental Example 3

### Dental Enamel Recalcification Promoting Effect, of Food or Beverage (Tablet) in the Present Invention

Several types of tablet were prepared for each Example in accordance with the mixing ratio shown in Table 2, the tablet comprising κ-carrageenan, ι-carrageenan and Furcelleran as an extract of the anhydrogalactose-containing red algae polysaccharide, and further in combination with xylitol and dicalcium phosphate:

Working Examples 4 to 6 (0.1% by weight of κ-carrageenan, ι-carrageenan and Furcelleran added, respectively);

Comparative Example 3 (no red algae extract added);

Comparative Example 4 (0.1% by weight of λ-carrageenan added).

TABLE 2

| | | Working |
|---|---|---|
| | | Examples 4 to 6, |
| | Comparative | Comparative |
| | Example 3 | Example 4 |
| Xylitol | 87.0% by weight | 87.0% by weight |
| Dietary Fiber | 4.0% by weight | 4.0% by weight |
| Emulsifier | 5.0% by weight | 5.0% by weight |
| Thickener | 3.8% by weight | 3.7% by weight |
| Dicalcium phosphate | 0.2% by weight | 0.2% by weight |
| Red algae polysaccharide | 0% by weight | 0.1% by weight |

The recalcification promoting effect of the tablet in Working Examples 4 to 6 and Comparative Examples 3 and 4 was tested in a manner similar to Experimental Example 2. The microradiographical (MR) results of each experimental dental enamel block were visually evaluated to be classified into five levels of recalcification, similarly to Experimental Example 1 of the recalcification promoting effect. The degrees of recalcification in said Working Examples 4 to 6 and Comparative Examples 3 and 4 were calculated as an average value among the 3 experimental dental enamel blocks for each Example, respectively, the value being graphically indicated, in FIG. 13.

The tablet in Comparative Example 3 (no red algae extract added) showed the recalcification degree of 1.67, while the tablet in Working Example 4 (0.1% of κ-carrageenan added) showed the recalcification degree of 2.67, the tablet in Working Example 5 (0.1% of ι-carrageenan added) showed the recalcification degree of 2.00, and the tablet in Working Example 6 (0.1% of Furcelleran added) showed the recalcification degree of 2.33. The tablet in Comparative Example 4 (0.1% of λ-carrageenan added) showed the recalcification degree of 1.67. This result proves that a red algae polysaccharide has a more intense recalcification promoting effect when it contains anhydrogalactose than otherwise.

The mixing ratios for Working Examples 7 to 20 are given below as working examples of the oral composition or the food or beverage related to the present invention (Unit: % by weight). Three types of red algae polysaccharide, κ-carrageenan, ι-carrageenan and Furcelleran, were used to form a red algae polysaccharide for e ach of these working examples.

Working Example 7 (Chewing Gum)

| | |
|---|---|
| Gum base | 20.0 |
| Sorbitol | 55.0 |
| Maltitol | 23.8 |
| Softener | 1.0 |
| Red algae polysaccharide | 0.2 |

Working Example 8 (Chewing Gum)

| | |
|---|---|
| Gum base | 20.0 |
| Xylitol | 55.0 |
| Maltitol | 22.5 |
| Softener | 1.0 |
| Dicalcium phosphate | 1.0 |
| Red algae polysaccharide | 0.5 |

Working Example 9 (Candy)

| | |
|---|---|
| Xylitol | 48.0 |
| Reduced maltose syrup | 36.5 |
| Dicalcium phosphate | 0.5 |
| Red algae polysaccharide | 0.5 |
| Fragrant material | 0.4 |
| Purified water | 14.1 |

Working Example 10 (Candy)

| | |
|---|---|
| Palatinit | 48.0 |
| Reduced maltose syrup | 36.0 |
| Red algae polysaccharide | 0.5 |
| Fragrant material | 0.4 |
| Purified water | 15.1 |

Working Example 11 (Tablet)

| | |
|---|---|
| Xylitol | 75.0 |
| Lactose | 20.9 |
| Glycerin fatty acid ester | 0.2 |
| Dicalcium phosphate | 0.05 |
| Red algae polysaccharide | 0.05 |
| Purified water | 3.8 |

Working Example 12 (Tablet)

| | |
|---|---|
| Xylitol | 75.0 |
| Palatinit | 20.0 |
| Glycerin fatty acid ester | 0.2 |
| Red algae polysaccharide | 0.5 |
| Purified water | 4.3 |

Working Example 13 (Chocolate)

| | |
|---|---|
| Cacaomas | 15.0 |
| Dry whole milk | 25.0 |
| Xylitol | 40.4 |
| Dicalcium phosphate | 0.5 |
| Cocoa butter | 18.0 |
| Emulsifier | 0.3 |
| Fragrant material | 0.3 |
| Red algae polysaccharide | 0.5 |

Working Example 14 (Chocolate)

| | |
|---|---|
| Cacaomas | 15.0 |
| Dry whole milk | 25.0 |
| Xylitol | 40.9 |
| Red algae polysaccharide | 0.5 |
| Cocoa butter | 18.0 |
| Emulsifier | 0.3 |
| Fragrant material | 0.3 |

Working Example 15 (Ice cream)

| | |
|---|---|
| Cream (fat percentage: 45%) | 25.0 |
| Milk (fat percentage: 3.7%) | 35.0 |
| Nonfat dry milk (sugar-free) | 24.3 |
| Xylitol | 10.4 |
| Dicalcium phosphate | 0.1 |
| Corn syrup | 4.4 |
| Stabilizer | 0.77 |
| Red algae polysaccharide | 0.03 |

Working Example :16 (Beverage)

| | |
|---|---|
| High-fructose corn syrup | 0.3 |
| Xylitol | 8.6 |
| Acidulant | 1.0 |
| Fragrant material | 0.4 |
| Dicalcium phosphate | 0.1 |
| Red algae polysaccharide | 0.3 |
| Purified water | 89.3 |

Working Example 17 (Dentifrice)

| | |
|---|---|
| Aluminum hydroxide | 35.0 |
| Anhydrous silicic acid | 15.0 |
| Xylitol | 10.0 |
| Dicalcium phosphate | 0.2 |
| Sodium lauryl sulphate | 1.0 |
| Fragrant material | 0.5 |
| Red algae polysaccharide | 0.3 |
| Purified water | 38.0 |

Working Example 18 (Mouth Washes)

| | |
|---|---|
| Xylitol | 20.0 |
| Glycerin | 10.0 |
| Sodium lauryl sulphate | 1.0 |
| Dicalcium phosphate | 0.5 |
| Fragrant material | 0.2 |
| Red algae polysaccharide | 0.5 |
| Purified water | 67.8 |

Working Example 19 (Mouth Washes)

| | |
|---|---|
| Xylitol | 7.4 |
| Red algae polysaccharide | 0.5 |
| Glycerin | 10.0 |
| Sodium lauryl sulphate | 1.5 |
| Fragrant material | 0.4 |
| Purified water | 80.2 |

Working Example 20 (Mouth Washes)

| | |
|---|---|
| Sorbitol | 7.4 |
| Dicalcium phosphate | 0.2 |
| Glycerin | 10.0 |
| Sodium lauryl sulphate | 1.5 |
| Fragrant material | 0.6 |
| Red algae polysaccharide | 0.3 |
| Purified water | 80.0 |

### BRIEF DESCRIPTION OF THE DRAWINGS

FIG. 1 is a view illustrating the microradiographical (MR) results immediately after the decalcified layer was formed in the experimental dental enamel block.

FIG. 2 is a view illustrating the in microradiographical (MR) results after the decalcified layer was formed in the experimental dental enamel block and then was treated with a recalcification solution.

FIG. 3 is a view illustrating the microradiographical (MR) results after the decalcified layer was formed in the experimental dental enamel block and then was treated with a recalcification solution containing 5% by weight of xylitol.

FIG. 4 is a view illustrating the microradiographical (MR) results after the decalcified layer was formed in the experimental dental, enamel block and then was treated with a recalcification solution containing 0.05% by weight of κ-carrageenan.

FIG. 5 is a view illustrating the microradiographical (MR) results after the decalcified layer was formed in the experimental dental enamel block and then was treated with a recalcification solution containing 0.05% by weight of κ-carrageenan, 5% by weight of xylitol and 0.02% by weight of dicalcium phosphate.

FIG. 6 is a view illustrating the microradiographical (MR) results after the decalcified layer was formed in the experimental dental enamel block and then was treated with a recalcification solution containing 0.05% by weight of λ-carrageenan.

FIG. 7 is a view illustrating the microradiographical (MR) results after the decalcified layer was formed in the experimental dental enamel block and then was treated with a recalcification solution containing 0.05% by weight of λ-carrageenan, 5% by weight of xylitol and 0.02% by weight of dicalcium phosphate.

FIG. 8 is a view illustrating the microradiographical (MR) results after the decalcified layer was formed in the experimental dental enamel block and then was treated with a recalcification solution containing 0.05% by weight of ι-carrageenan.

FIG. 9 is a view illustrating the microradiographical (MR) results after the decalcified layer was formed in the experimental dental enamel block and then was treated with a recalcification solution containing 0.05% by weight of ι-carrageenan, 5% by weight of xylitol. and 0.02% by weight of dicalcium phosphate.

FIG. 10 is a view illustrating the microradiographical (MR) results after the decalcified layer was formed in the experimental dental enamel block and then was treated with a recalcification solution containing 0.05% by weight of Furcelleran.

FIG. 11 is a view illustrating the microradiographical (MR) results after the decalcified layer was formed in the experimental dental enamel block and then was treated with a recalcification solution containing 0.05% by weight of Furcelleran, 5% by weight of xylitol and 0.02% by weight of dicalcium phosphate.

FIG. 12 is a chart showing the microradiographical (MR) results after the decalcified layer was formed in the experimental dental enamel block and then was treated with each chewing gum extract prepared by means of the recalcification solution.

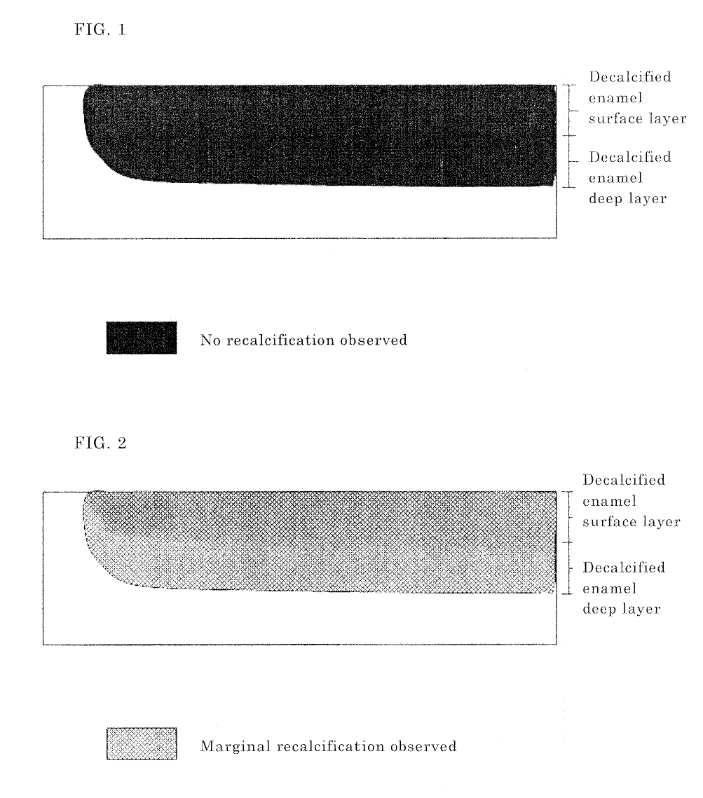

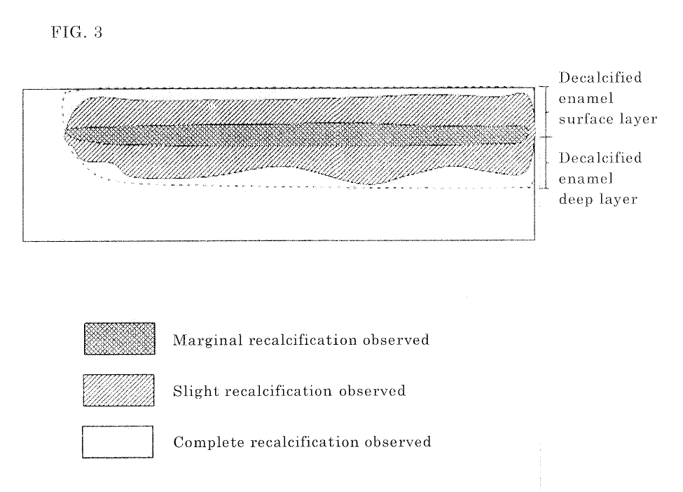

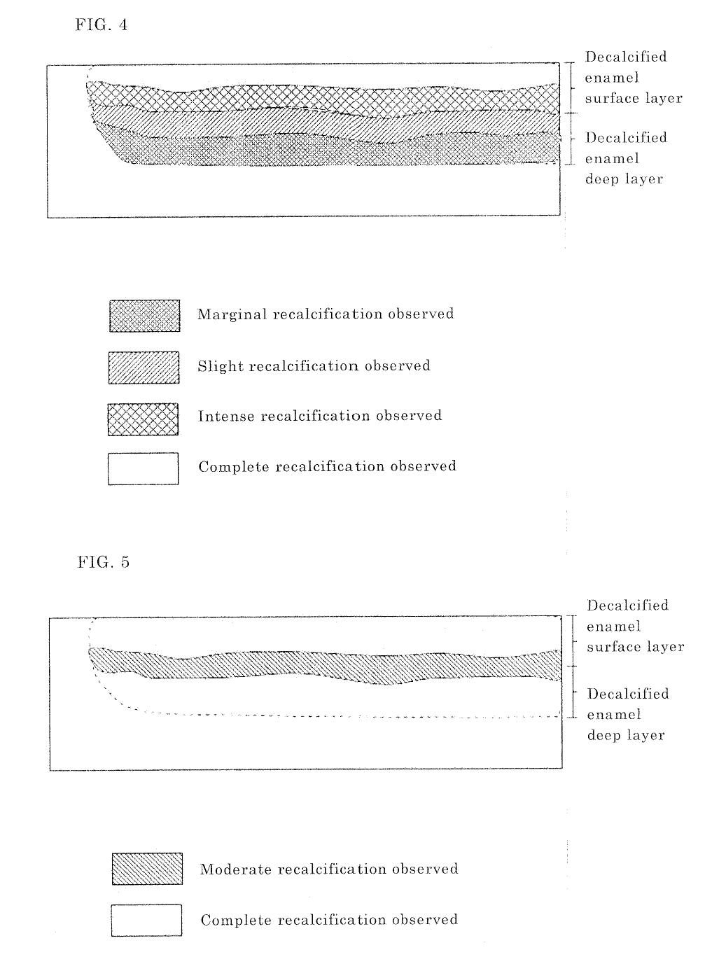

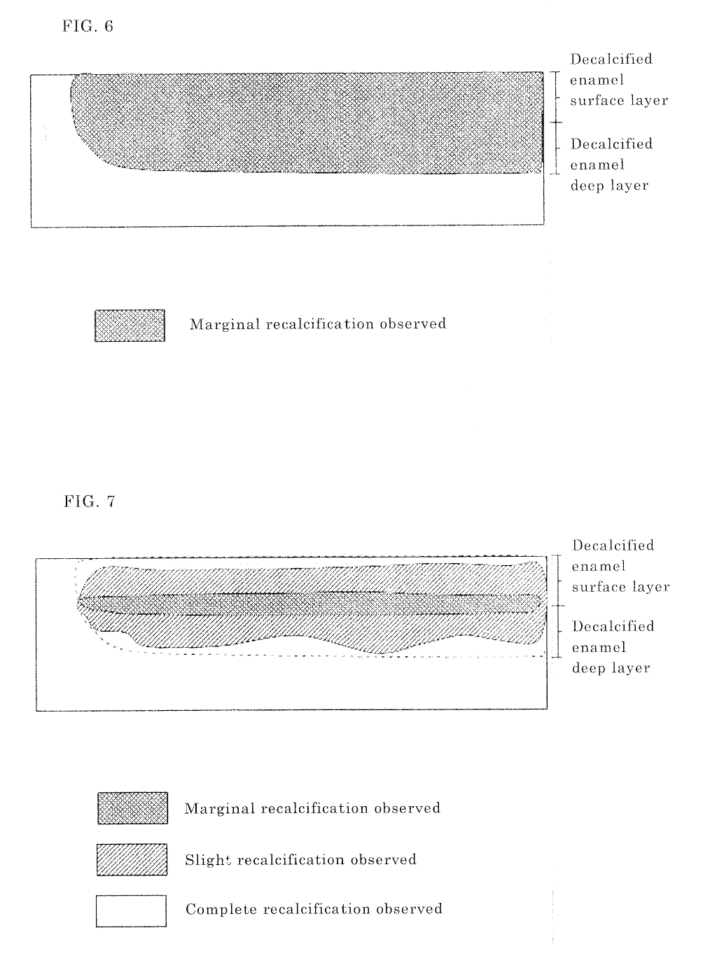

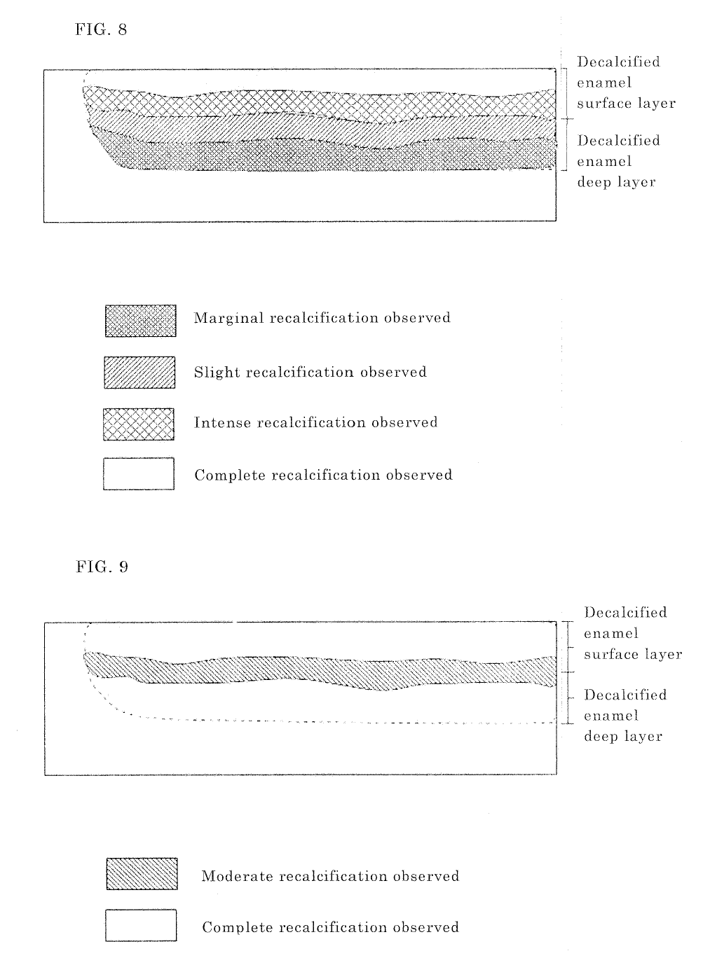

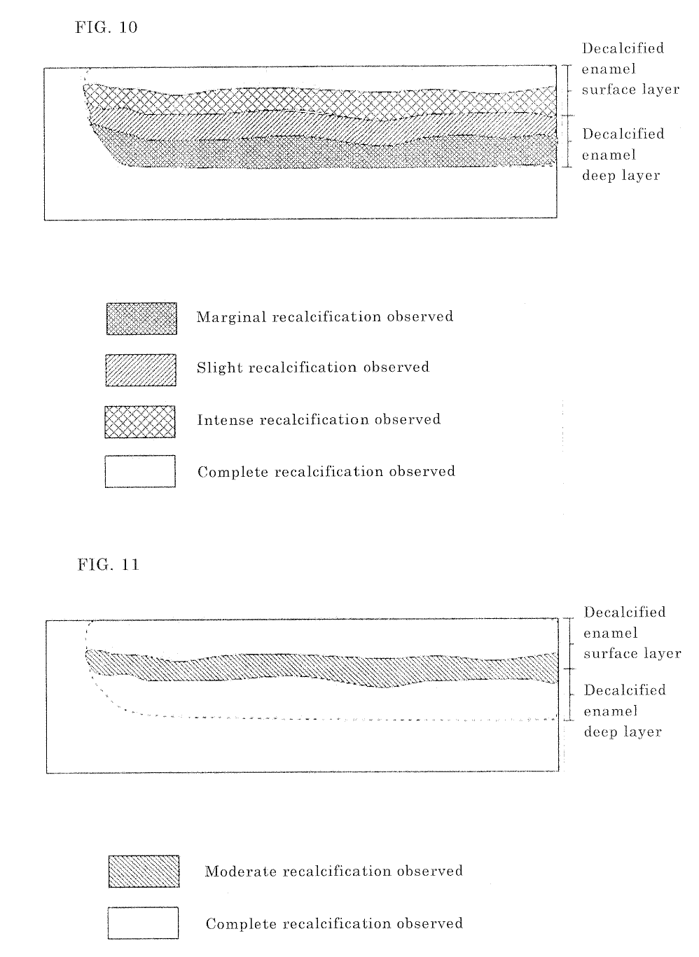

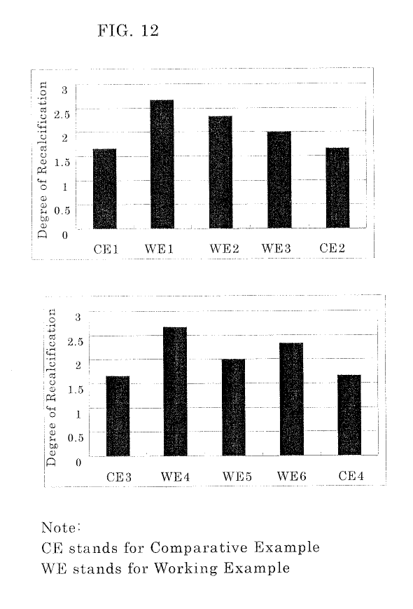

1. A dental enamel recalcification accelerator comprising, as an active ingredient, anhydrogalactose-containing red algae and/or an extract thereof.

2. A dental enamel recalcification accelerator comprising, as an active ingredient, anhydrogalactose-containing red algae and/or an extract thereof, and xylitol and/or dicalcium phosphate.

3. The dental enamel recalcification accelerator according to claim 1, wherein the extract of anhydrogalactose-containing red algae is at least one polysaccharide selected from the group consisting of κ-carrageenan, ι-carrageenan and Furcelleran.

4. An oral composition containing the dental enamel recalcification accelerator according to claim 1.

5. The oral composition according to claim 4, wherein an indication is attached to the effect that recalcification of teeth will be enhanced.

6. A food or beverage containing the dental enamel recalcification accelerator according to claim 1.

7. The food or beverage according to claim 6, wherein an indication is attached to the effect that the teeth recalcification will be enhanced.

8. The dental enamel recalcification accelerator according to claim 2, wherein the extract of anhydrogalactose-containing red algae is at least one polysaccharide selected from the group consisting of κ-carrageenan, ι-carrageenan and Furcelleran.

9. An oral composition containing the dental enamel recalcification accelerator according to claim 2.

10. An oral composition containing the dental enamel recalcification accelerator according to claim 3.

11. A food or beverage containing the dental enamel recalcification accelerator according to claim 2.

12. A food or beverage containing the dental enamel recalcification accelerator according to claim 3.
